(19) United States
(12) Patent Application Publication
(10) Pub. No.: US 2016/0346059 A1
(43) Pub. Date: Dec. 1, 2016

(54) Skin Marking Device

(71) Applicant: Jeffery C. McNeely, Lubbock, TX (US)

(72) Inventor: Jeffery C. McNeely, Lubbock, TX (US)

(21) Appl. No.: 14/725,770

(22) Filed: May 29, 2015

(51) Int. Cl. A61B 90/00; A61B 6/00; A61B 6/12

(52) U.S. Cl. CPC A61B 90/39; A61B 6/12; A61B 6/4441

(57) ABSTRACT

A surgical skin marking device comprising a handle, a marking agent, a marking tip extending forwardly of the handle and configured to apply a marking agent to the skin when contacted with the skin by a user, and a radiopaque pointer that is positioned in a defined relationship relative to the handle and is proximally disposed relative to the marking tip to facilitate more accurate skin marking by a surgeon during preoperative and intra-operative procedures. A method of using the subject surgical skin marking device is also disclosed.

### BACKGROUND OF THE INVENTION

1. Field of the Invention

This invention relates to a device intended for medical use and, more particularly, to a skin marking device for preoperative and intraoperative use, or medical teaching. The invention desirably has a tip end designed and configured to be visible (“radiopaque”) during radiographic imaging of bone. The subject device can be used, for example, in conjunction with a Zeiss Scan, X-Ray, C-Arm or Mini C-Arm, and will enable a physician or surgeon to observe placement of the tip end of the skin marking device in relation to skeletal elements. This can be done for purposes such as marking the skin surface at positions that are proximal to bones, joints, bone tumors, fractures, dislocations, other skeletal abnormalities or anomalies, and the like, that are of interest before or during a surgical procedure, or for another medically related reason.

Another worthwhile use of the subject invention is for instructional purposes during training of physicians and other medical professionals. The present invention is believed to improve surgical efficiency and precision for bone-related procedures while also reducing the cumulative time that the hands of a surgeon are subjected to radiation exposure. The subject device is not intended or suitable for use with nuclear magnetic resonance (NMR) or computerized tomography (CT) imaging.

2. Description of Related Art

Both non-sterile and sterile surgical skin markers are well known and commercially available. Difficulties have previously been experienced, however, when conventional skin markers have been used in attempts to precisely identify and mark on the skin surface the positions where bones, joints, fractures, tumors or other skeletal abnormalities lie beneath the skin, particularly in areas of the body that are not easily palpated. Because conventional skin markers typically have no metal components, surgeons cannot see the actual position of the skin marker while using a radiographic imaging device such as a Zeiss Scan, C-Arm, Mini C-Arm or X-Ray to observe bones either preoperatively or intra-operatively.

During radiographic imaging, a surgeon will often ask a surgical tech or other assistant to pick up a radiopaque metal instrument off the back surgical table, place the instrument on a patient's skin and move it around as instructed by the surgeon until the instrument is properly positioned in a desired area relative to the skeletal structure being observed, after which the surgeon or tech will mark the area alongside the radiopaque instrument with a skin marking device that is not radiopaque and is accordingly invisible in the images obtained from the radiographic imaging device. Because this marking procedure is typically done using a straight sided instrument that does not follow the curvature of a joint, fracture or tumor, etc., it can result, for example, in misplaced or overly long incisions where the skin markings are imprecisely applied in relation to the skeletal features that are under examination for purposes of reduction, excision, rearrangement or repair. Other factors such as the portion of the anatomy under examination, the particular radiographic imaging device being used, the skill of the surgical tech, and the configuration of the radiopaque instrument selected can also affect the effectiveness of such procedures when using conventional surgical skin markers.

Previously issued patents disclosing surgical skin markers or other markers for diagnostic or surgical procedures include, for example, U.S. Pat. Nos. 8,152,401; 6,972,022; 6,923,816; 6,056,737; 5,6654,092; and 4,916,170.

### SUMMARY OF THE INVENTION

A surgical skin marker and method of use for preoperative or intra-operative use are disclosed here that embody and utilize a radiopaque pointer that enables a surgeon to plainly see on a radiographic imaging screen associated with a Zeiss Scan, an X-Ray machine, a C-Arm or a Mini C-Arm, the front end portion of the subject skin marker and to manipulate the marker in his or her own hand to mark the skin while viewing the radiopaque portion of the marker on the imaging screen in real time. The subject device enables a surgeon to more conveniently, quickly and reliably position a radiopaque front end portion of the subject surgical skin marker and an associated marker tip proximally to a desired position on the skin. With this tool a surgeon can, for example, view the actual position of normal, fractured, dislocated, diseased or previously repaired bones; view plates, pins, screws or other hardware; and view the positions of any embedded foreign object in real time. The surgeon can also quickly, conveniently and reliably mark points, positions or outlines on the skin that correspond to the subdermal positions of such features or the boundaries of incisions to be made during a procedure.

The radiopaque pointer enables the user to hold the front end portion proximally to a surface portion of a patient's skin and mark the skin in a position that is more closely associated with and indicative of the actual position of a subdermal bone, joint, fracture, tumor or other bone abnormality in relation to the marking on the skin. With the new surgical skin marker of the invention, a physician or surgeon can see the radiopaque pointer at or near the front end of the marker on the bone imaging device and can mark the skin directly without the extra steps attendant to use of another instrument from the surgical table. It also dramatically increases the accuracy of following the contour, shape and curvature of the anatomical structures being viewed by the surgeon on the radiographic imaging screen.

The surgical skin marker of the invention desirably comprises a handle, a marking agent, a marking tip extending forwardly of the handle and configured to apply a marking agent to the skin when contacted with the skin by a user, and a radiopaque pointer that is aligned with the handle and proximally disposed relative to the marking tip.

According to one embodiment of the invention, the radiopaque pointer is an integral part of the handle such as by inlaying or insert molding the radiopaque pointer into the handle. According to another embodiment of the invention, the radiopaque pointer is directly attachable to and frictionally engageable with the handle. According to another embodiment of the invention, the radiopaque pointer is attached to a member such as split collar that frictionally engages the handle. According to another embodiment of the invention, the radiopaque pointer is a position indicator in the form of a radiopaque dot or spot disposed on the handle in a visible position (when viewed through a radiographic image display) that is proximal to and behind the marking tip. According to another embodiment of the invention, the radiopaque pointer comprises a radiopaque surface layer that is coated or deposited onto, or laminated or adhered to a substrate having a pressure sensitive adhesive that is selectively attachable to a portion of the handle in a position that is proximal to and slightly behind the marking tip. According to another embodiment of the invention, the position of the radiopaque pointer and the length of the handle can vary, and are optionally independently adjustable relative to the marking tip. According to another embodiment of the invention, the alignment of the radiopaque pointer and marking tip are selectively adjustable relative to the handle. According to another embodiment of the invention, the length of the handle is selectively adjustable or is otherwise configured to avoid prolonged or excessive cumulative exposure of the surgeon's hands to potentially harmful radiation. According to another embodiment of the invention, the surgical skin marker is sterile. According to another embodiment of the invention, the marking tip and marking agent are sterile. According to another embodiment of the invention, the radiopaque pointer is sterile. According to another embodiment of the invention, the handle, radiopaque pointer and marking tip are disposable. According to another embodiment of the invention, the radiopaque pointer and marking tip are disposable.

An inventive method is also disclosed that desirably comprises providing a surgical skin marker further comprising a handle, a marking agent, a marking tip extending forwardly of the handle and configured to apply a marking agent to the skin when contacted with the skin by a user, and a radiopaque pointer that is disposed on the handle in a position proximal to the marking tip; placing the surgical skin marker in a position within a viewing field of an imaging screen of a radiographic imaging device, which position causes the radiopaque pointer to be proximal to the skin surface and the marking tip; and then marking the skin as desired while viewing in real time the skeletal elements of a patient that are of interest for achieving a desired purpose, surgical objective or other medical treatment.

The desired purpose can include, for example and without limitation, to outline the positions of normal or abnormal bones, joints, fractures, tumors or other skeletal anomalies or abnormalities appearing within the viewing field (especially those presenting in an irregular pattern as opposed to a straight line); to trace the positions of fractures, dislocations or tumors; to identify surgical incision points and boundaries on the skin surface; to visualize and mark skeletal features that cannot be palpated (such as vertebrae, in the treatment of scoliosis); to determine and mark the position of foreign objects embedded in the body or bones; to repair or replace previously placed hardware that has failed or become ineffective; to assist intra-operatively with percutaneous pinning (such as with comminuted fractures), and to identify skeletal elements of interest while instructing students in a medical teaching environment.

The shape and size of the radiopaque pointers can be determined as appropriate for a particular application as desired. Suitable materials for use as radiopaque pointers for use in the subject surgical skin markers can include, for example and without limitation: metal strips, inserts, tips or attachable collars; powdered or slurried radiopaque compounds used as coatings or disposed inside chambers or viewing windows in the handles that are visible to a surgeon using a radiographic viewing instrument, or radiopaque fluids or suspensions that can be painted, coated, coextruded, or vapor deposited onto a portion of the handle of a surgical skin marker and dried in placed to create a viewable radiopaque pointer. Similarly, any other radiopaque substance or article that can be molded, glued, or attached to the handle of a surgical skin marker, or coated onto the handle, or applied to another substrate that is attachable to the handle, can also be used in making the surgical skin markers of the invention. The length of the handle portion of the subject surgical skin markers can be made in different fixed lengths, or can be telescoping or otherwise variable, recognizing that surgeons or other uses will desirably want to avoid or minimize exposure of their hands to potentially harmful radiation or electromagnetic waves while holding and manipulating a surgical skin marker having a radiopaque pointer that is disposed within the field of view.

These and other features, objects and advantages of the present invention will become better understood from a consideration of the following detailed description of the preferred embodiments and appended claims in conjunction with the drawings.

### BRIEF DESCRIPTION OF THE DRAWINGS

The system and method of the invention are further described and explained in relation to the following drawings wherein:

FIG. 1 is a simulated top plan view of the left hand and wrist of a patient as viewed with a camera, and is labeled as “Prior Art;”

FIG. 2 is a simulated top plan view of a radiographic image of the left wrist of a patient as in FIG. 1 when viewed using a radiographic imaging device, and is also labeled as “Prior Art;”

FIG. 3 is a simulated top plan view of a radiographic image of the left wrist of a patient as in FIG. 2 with one embodiment of a radiopaque pointer of a skin marking device of the invention superimposed over a portion of the wrist in relation to a radiopaque marking applied by a surgeon to the patient's skin by manipulating a front tip of the skin marking device against the patient's skin at the desired location;

FIG. 4 is a simulated top plan view image of the left hand and wrist of the patient as in FIG. 1, wherein the marking tip of a skin marking device of the invention has been used by a surgeon to mark the skin surface with a radiopaque substance as in FIG. 3 to define preferred surgical incision boundaries by a physician or surgeon manipulating a radiopaque pointer of the subject invention while viewing the image as in FIG. 3;

FIG. 5 is a simulated top plan view of the left hand and wrist of a patient as in FIG. 1 but having the bones of the palm and wrist drawn on the skin surface of the back of the left palm and wrist by a physician or surgeon while viewing a radiographic image of the skeletal components in that part of the patient's anatomy as might be done, for example, as part of a teaching exercise;

FIG. 6 is a front perspective view of one embodiment of the skin marking device of the invention in which the radiopaque pointer is a metallic strip attached in generally parallel relation to a forwardly extending end of the handle;

FIG. 7 is an enlarged front perspective view of one embodiment of a radiopaque pointer that is frictionally engageable with a handle portion of a surgical skin marker and can be adjustably positioned along a handle portion of a surgical skin marker as shown, for example, in FIG. 3;

FIG. 8 is an enlarged front perspective view of an embodiment of a surgical skin marking device of the invention, partially broken away, wherein the radiopaque pointer is a similarly configured but larger metal insert than in FIG. 6 that is disposed just behind the marker tip so as to make the radiopaque pointer more easily visible;

FIG. 9 is a front perspective view, partially broken away, of an embodiment of a skin marking device similar to that of FIG. 3 wherein the radiopaque pointer is a dot or spot disposed in a visible position on the handle proximally behind the marker tip;

FIG. 10 is a front perspective view of an embodiment of the skin marking device of the invention wherein the radiopaque pointer is a radiopaque band that wraps or surrounds the handle proximally behind the marker tip;

FIG. 11 is a front perspective view of a toroidal roll of radiopaque elastomeric material having an open center, the roll being configured to slip over and sheathe the front portion of a conventional surgical skin marker not otherwise having a radiopaque pointer, so that it can be unrolled onto the marker to make the portion behind the marker tip radiopaque;

FIG. 12 is a cross-sectional elevation view taken along line 12-12 of FIG. 11;

FIG. 13 is a front perspective view of the toroidal roll of FIGS. 11 and 12 being applied to the front portion of a conventional surgical skin marker;

FIG. 14 is front elevation view of a rolled strip of cellular material having a metalized film layer applied over it, the rolled strip being adhesively attachable to the surface of a front portion of a conventional surgical skin marker to make a front portion of the marker radiopaque; and

FIG. 15 is a front perspective view of a conventional surgical skin marker having the radiopaque strip of cellular material of FIG. 14 wrapped around it to make the front portion of the marker radiopaque.

### DESCRIPTION OF PREFERRED EMBODIMENT

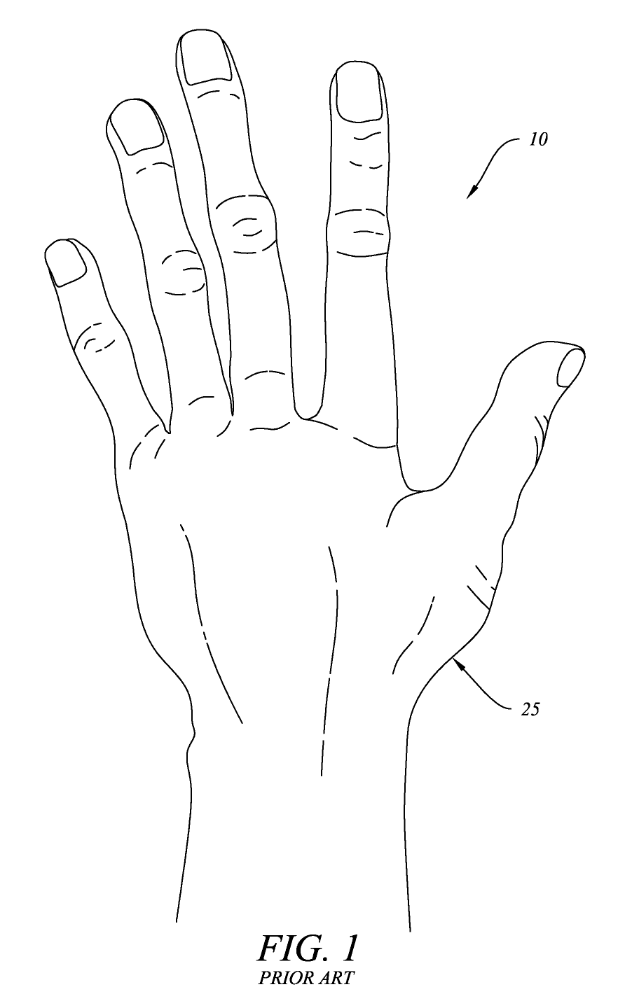

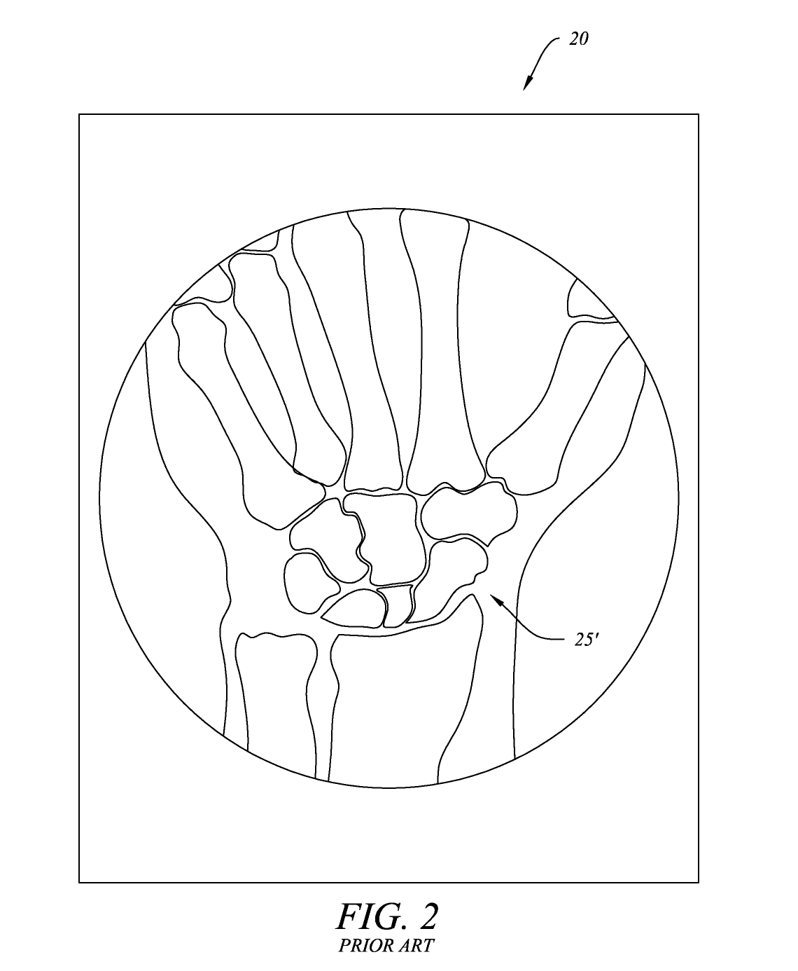

Referring to FIG. 1, the left hand 10 and wrist 25 of a human are depicted diagrammatically for use in explaining the utility of the subject invention. In FIG. 2, the skeletal structure of the left palm and wrist 25′ as enlarged from FIG. 1 are depicted diagrammatically to show how they might appear within the viewing field 20 of a conventional imaging screen that is operatively linked to a conventional radiographic imaging device as described above. Both FIGS. 1 and 2 are labeled as “Prior Art” because they do not comprise elements of the invention.

Referring to FIG. 3, viewing field 20 and the skeletal structure of the left palm and wrist 25′ are again depicted as in FIG. 2 but with a surgical skin marker 40 modified to include a radiopaque pointer 42′ in accordance with the invention. With the exception of the radiopaque pointer 42, which is visible with viewing field 20, other portions of surgical skin marker 40 are depicted in broken lines because they are not visible to the user where hidden behind or beneath the imaging screen of the radiographic imaging device. Also visible in FIG. 3 is a mark 30 produced on the skin of the patient by marker tip 44 when surgical skin marker 40 is manipulated by the surgeon (or other user). Although mark 30 is linear, it should be appreciated by those of skill in the art reading this disclosure that an advantage of the apparatus and method disclosed here is the capability of marking the skin while observing the skeletal features in real time to outline the shape and position of non-linear features of the skeletal anatomy such as fractures, comminuted fractures, joints, dislocations, and the like, as well as embedded projectiles or other foreign objects that have penetrated the skin.

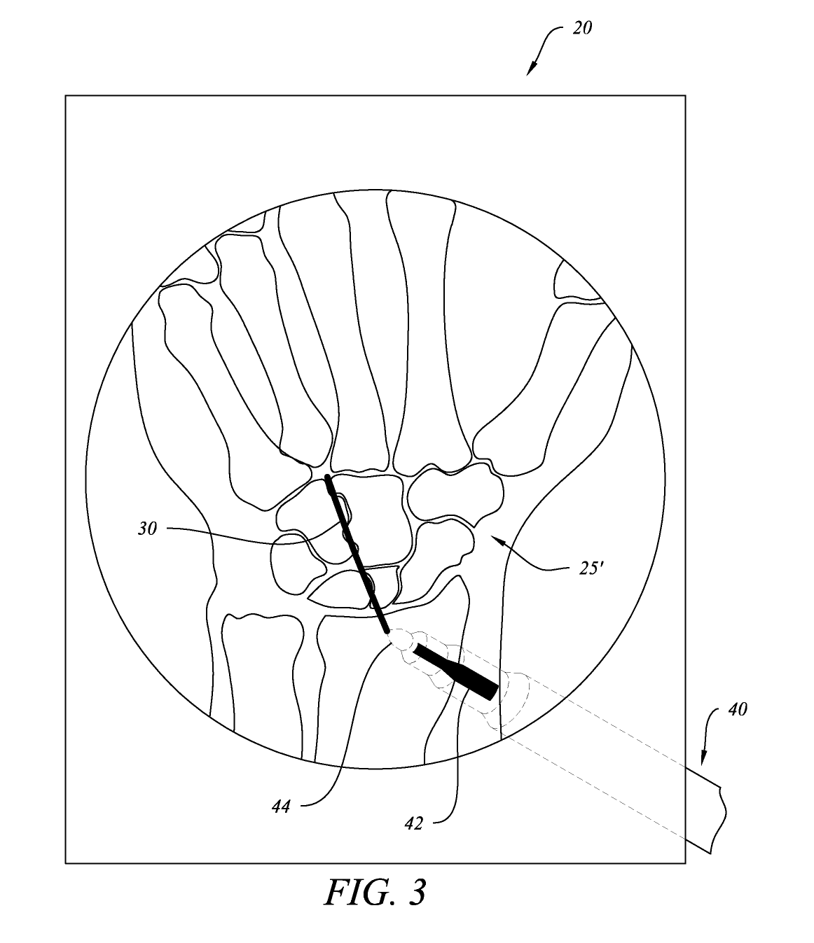

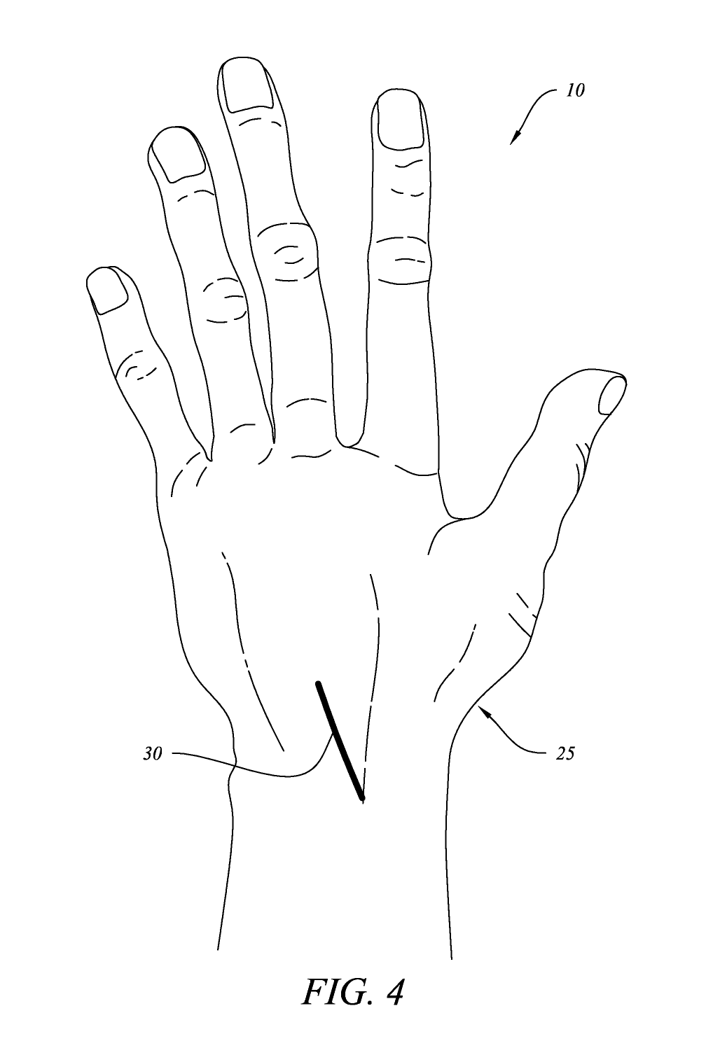

Referring to FIG. 4, radiopaque mark 30 produced by marker tip 44 as disclosed above appears on the skin of the patient's palm and wrist when viewed directly following placement of the radiopaque mark on the skin by the surgeon in real time while the surgeon was actually viewing the skeletal elements 25′ of the left palm and wrist of the patient within viewing field 20 of the imaging screen of the radiographic viewing device. Such a mark 30 might be made, for example, to define the boundaries of an incision for a surgical procedure to be performed on the patient.

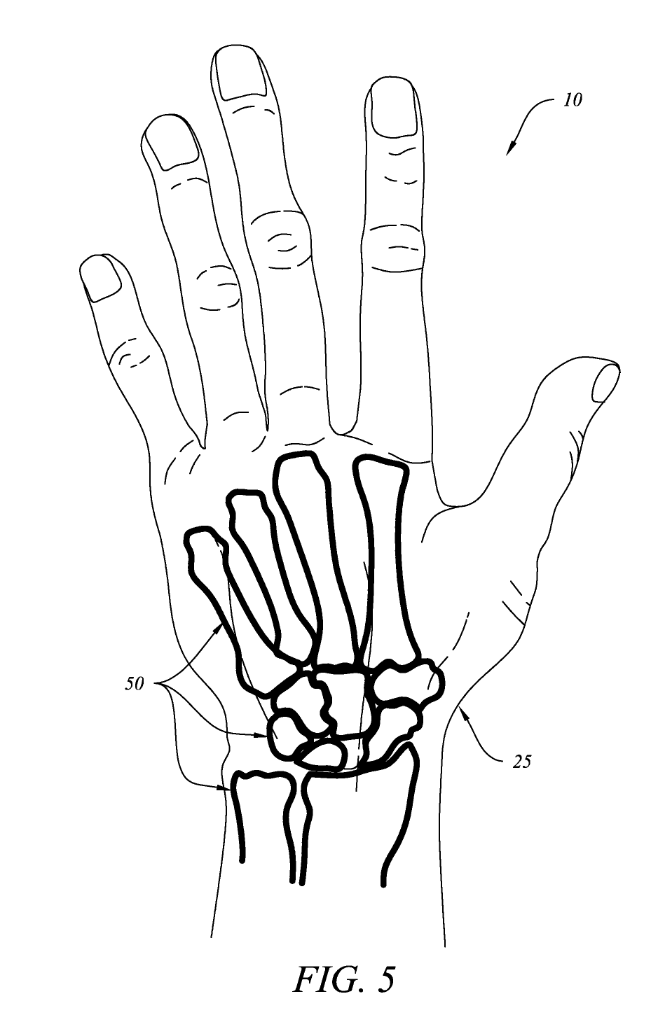

Referring to FIG. 5, left hand and wrist 10 of the patient are shown after a surgeon has used a modified surgical skin marker of the invention to outline a substantial portion of the skeletal elements of the left palm, wrist, and lower forearm in real time while simultaneously viewing the same skeletal elements disposed beneath the patient's skin within viewing field 20 (as in FIG. 3) associated with a radiographic imaging device such as, for example, a Zeiss Scan, an X-Ray machine, a C-Arm or a Mini C-Arm.

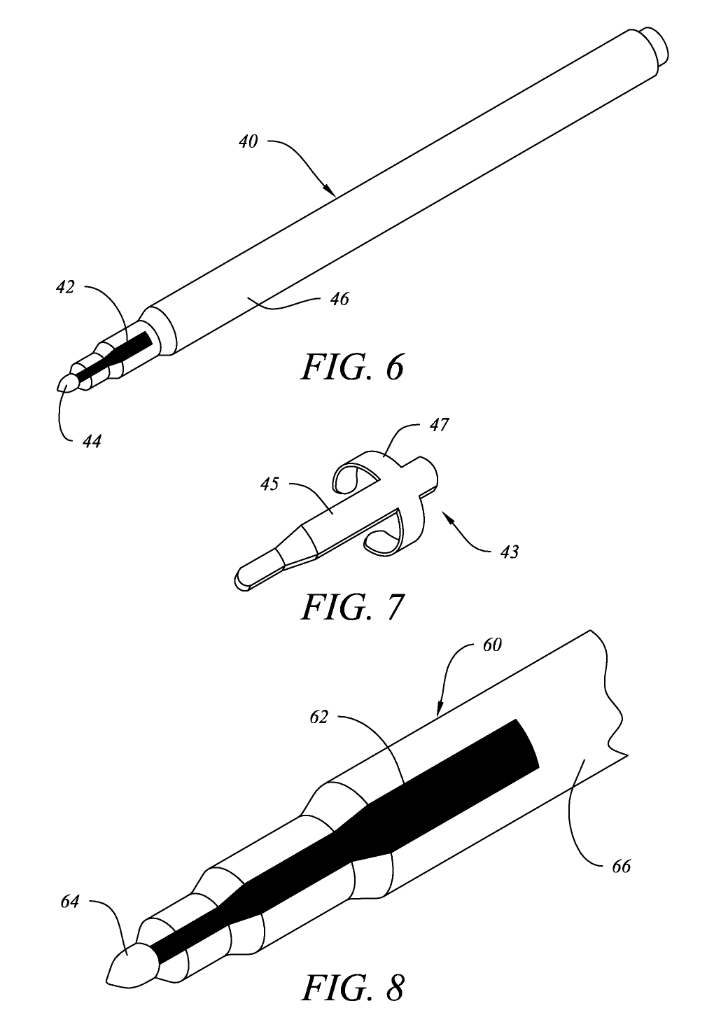

Referring to FIG. 6, the full length of modified surgical skin marker 40 is disclosed. By use of the term “modified,” reference is made to the provisions of a radiopaque pointer 42 that is not known to be present in commercially available surgical skin markers. Surgical skin marker 40 also desirably comprises a reservoir of radiopaque fluid that is applied to a patient's skin by contacting the skin of a patient with marker tip 44 and then moving marker tip 44 in a desired direction or directions over the skin surface while maintaining contact with the skin to apply one or more radiopaque, preferably indelible, marks to the skin in the desired location or locations. The present device is intended for preoperative or intra-operative use by a surgeon in accordance with the method of the invention while simultaneously viewing in real time the skeletal elements disposed beneath the skin of a patient, the position of the radiopaque pointer 42, and the radiopaque markings being made on the skin as modified surgical skin marker 40 is manipulated by the surgeon.

Those of ordinary skill in the art will appreciate upon reading this disclosure in relation to the accompanying drawing figures that the marker sizes and types can vary within the scope of the invention, as can the length of the marker handles 46, the size and type of marker tip 44 and the composition of the radiopaque marking agent disposed inside surgical skin marker 40. Similarly, the structure, configuration, composition and method of attachment of radiopaque pointers 42 can vary widely depending on many factors, including for example and without limitation, those depicted in FIGS. 6-15. Some embodiments of the invention utilize conventional, commercially available skin marking devices that are further modified to incorporate a selectively attachable radiopaque pointer as disclosed herein, while other embodiments can be specifically designed to incorporate a radiopaque pointer as an integral part of the device.

Referring to FIG. 7, radiopaque pointer 43 is disclosed that is desirably made of malleable sheet metal or another similarly effective radiopaque material and desirably comprises an elongate pointer portion 45 and an arcuate split ring collar portion 47 that is selectively attachable to a conventional surgical skin marker. Referring to FIG. 8, another radiopaque pointer 62 is disclosed in conjunction with surgical skin marker 60 having handle 66 and marker tip 64. Radiopaque 62 can be made, for example and without limitation, of metal that is insert molded into a plastic handle 66, or can be a metallized layer disposed on the upwardly facing surface of a substrate permanently affixed to handle 66, or can be a radiopaque coating or sticker that has been applied in a position substantially parallel to the longitudinal axis through handle 66 to assist a user in recognizing in what direction marker tip 64 is facing at any particular time as surgical skin marker 60 is manipulated by the user within a viewing field 20 as described above. By comparison of radiopaque pointer 42 in FIG. 6 with radiopaque pointer 62 in FIG. 8, it is evident that the overall size and dimensions of the radiopaque pointer can vary in different surgical skin markers and in surgical skin markers intended for different uses or in different conditions.

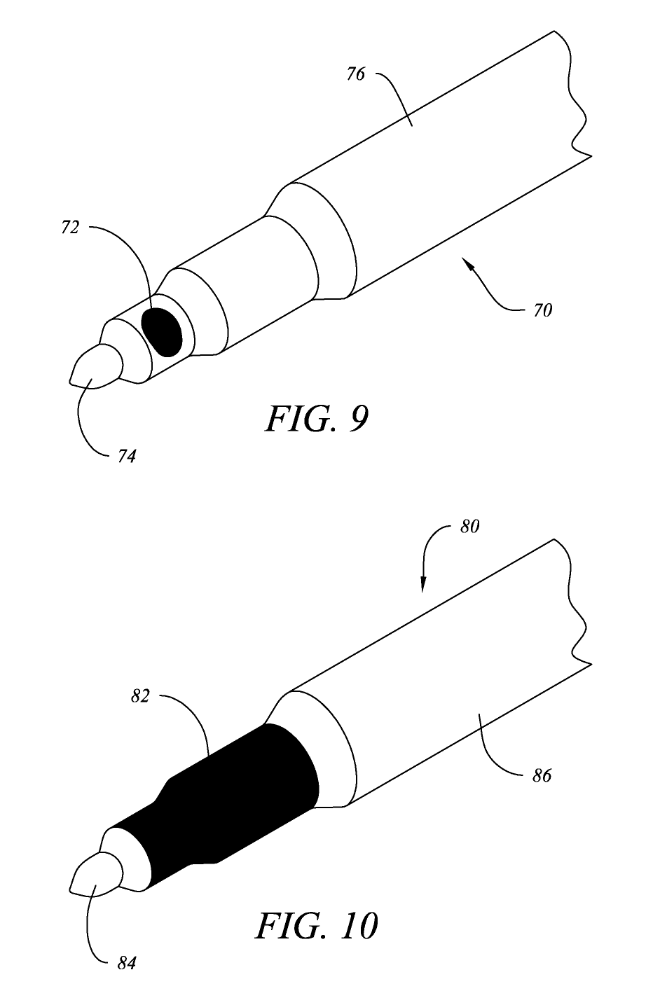

Referring to FIG. 9, a surgical skin marker 70 is disclosed that comprises handle 76, marker tip 74 and a spot or dot 72 that serves as the radiopaque pointer and is desirably disposed in a position that is proximal to marker tip 74. With such a device, it is important that the user rotate the marker to a position where dot 72 is visible within a viewing field such as viewing field 20 described above.

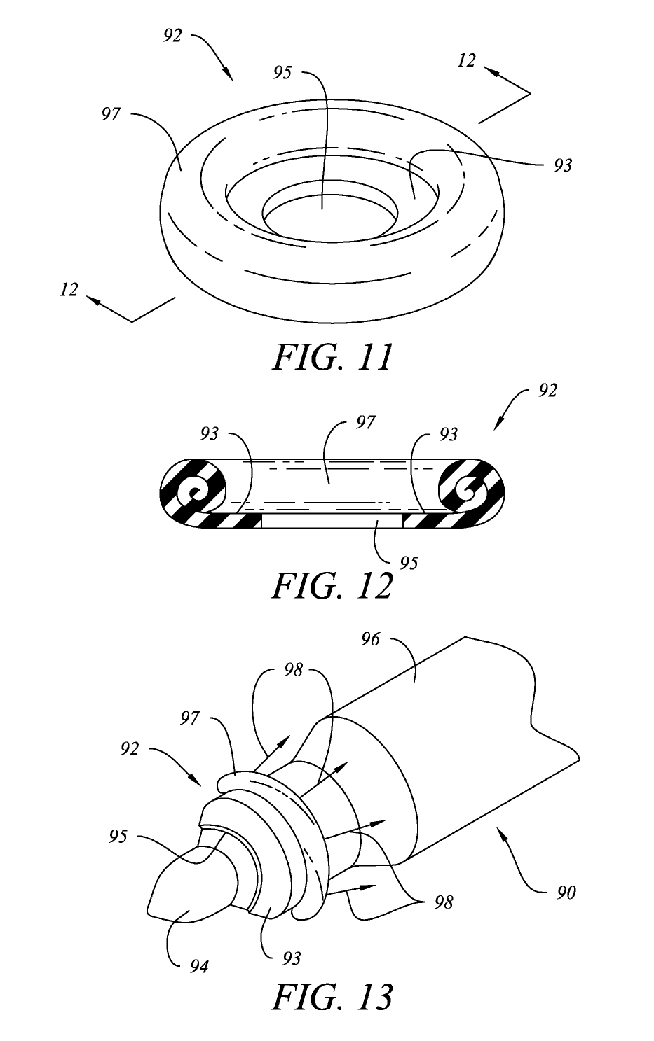

Referring to FIGS. 11-13, a radiopaque pointer is disclosed in the form of a toroidal roll of radiopaque elastomeric material that can be applied to a conventional surgical skin marker 90 comprising marker tip 94 for use in the method of the invention. Center portion 93 of toroidal roll 97 of radiographic pointer 92 is provided with a centrally disposed aperture 95 that can be stored in a sterile, small-volume package (not shown) prior to use and then placed over and around marker tip 94, after which toroidal roll 97 is unrolled upwardly over handle 96 as indicated by arrows 98 to ready the modified surgical skin marker 90 as needed for use in the method of the invention.

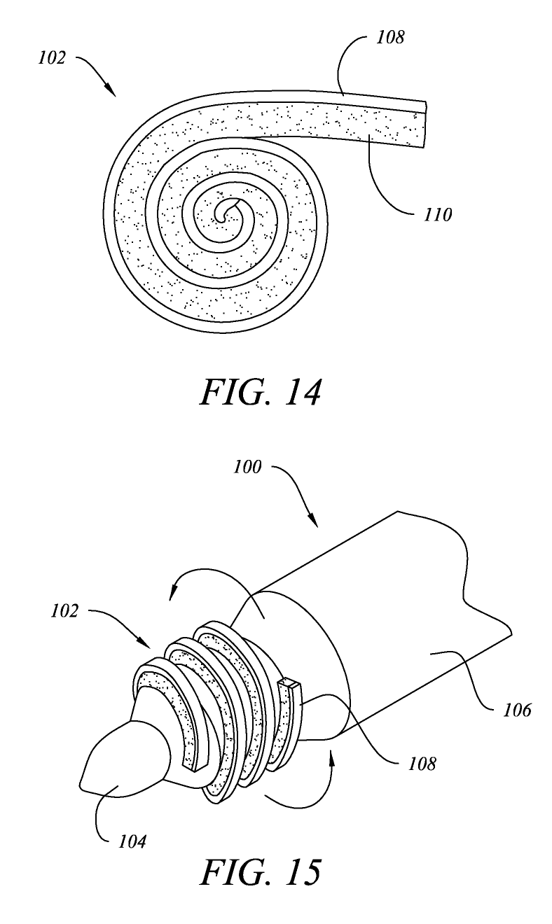

Referring to FIGS. 14 and 15, another embodiment of a modified surgical skin marker 100 is shown in which roll 102 of a pliable, adhesively attachable strip 110 having upwardly facing metalized or otherwise coated layer 108 is wrapped around the front portion of handle 106 of surgical skin marker 100 to render the front portion of the modified surgical skin marker radiopaque for use in the method of the invention as previously described. (It should be appreciated that the thicknesses of adhesively attachable strip 110 and layer 108 are exaggerated for illustrative purposes and are not necessarily representative of the actual thicknesses that might be used.)

Those of ordinary skill in the art will also appreciate upon reading this specification and the description of embodiments disclosed herein, and in the context of the accompanying drawings, that modifications and alterations to the apparatus and methods expressly disclosed in this application may be made within the scope of the invention. Applicant intends that the scope of the invention disclosed herein be limited only by the broadest interpretation of the appended claims to which Applicant is legally entitled without the exercise of hindsight after having read this disclosure.

What is claimed is:

1. A radiopaque pointer attachable to a surgical skin marker having a marker tip, the radiopaque pointer being configured to enable a healthcare professional to determine the location of the marker tip on an imaging screen of a radiographic imaging device, to manually manipulate the surgical skin marking device relative to the skin of a patient while viewing the radiopaque pointer in real time on the imaging screen to place the marking tip proximally to a desired position on the skin that corresponds to an observed percutaneous position of a bone, joint, dislocation, fracture, tumor or other skeletal element, condition or anomaly, and to mark the skin with the marking tip in a desired location as determined using the radiopaque pointer.

2. A skin marking device comprising a radiopaque pointer disposed proximally to and rearwardly of a marker tip, the radiopaque pointer being visible to a physician or surgeon within a viewing field of a radiographic imaging device, whereby the physician or surgeon can manipulate the device to mark the skin of a patient directly while viewing both the radiopaque pointer and the subdermal skeletal elements of the patient.

3. A surgical skin marking device for real time preoperative or intraoperative use by a surgeon facilitating marking of the skin of a patient relative to observed subdermal skeletal features, the device comprising a handle, a marker tip extending forwardly of the handle, a marking agent communicating with the marker tip and formulated to indelibly mark the skin when the marker tip is contacted with the skin by a user, and a radiopaque positional indicator element that is disposed in a predetermined positional relationship to the handle and to the marker tip, whereby the radiopaque positional indicator element is viewable by the user in juxtaposition to the observed subdermal skeletal features while the user manipulates the marker tip to apply the marking agent to the skin.

4. The surgical skin marking device of claim 3 wherein the radiopaque positional indicator element comprises a radiopaque pointer.

5. The surgical skin marking device of claim 3 wherein the radiopaque positional indicator element is connected to the handle.

6. The surgical skin marking device of claim 5 wherein the radiopaque positional indicator element is insert-molded into the handle.

7. The surgical skin marking device of claim 3 wherein the radiopaque positional indicator element is adjustably engageable with the handle.

8. The surgical skin marking device of claim 3 wherein the radiopaque positional indicator element is frictionally engageable with the handle.

9. The surgical skin marking device of claim 5 wherein the radiopaque positional indicator element is a metalized strip that is attachable to the handle.

10. The surgical skin marking device of claim 8 wherein the radiopaque positional indicator element is attached to a collar that is attachable to the handle.

11. The surgical skin marking device of claim 3 wherein the radiopaque positional indicator element is a radiopaque dot or spot disposed on the handle in a position that is visible on an imaging screen of a radiographic imaging machine and is proximal to and behind the marker tip.

12. The surgical skin marking device of claim 3 wherein the radiopaque positional indicator element is selectively adjustable relative to the marking tip.

13. The surgical skin marking device of claim 3 having an adjustable length handle.

14. A radiopaque pointer for a surgical skin marker, the radiopaque pointer comprising a substrate or composition that is selectively attachable to surgical skin marker.

15. The radiopaque pointer of claim 14 comprising a metallic outer surface.

16. The radiopaque pointer of claim 15 comprising a metallic strip.

17. The radiopaque pointer of claim 15 comprising a metallic collar.

18. The radiopaque pointer of claim 15 comprising a metallic clip.

19. The radiopaque pointer of claim 15 comprising a metallic coating.

20. A method for marking the skin of a medical patient, the method comprising:
providing a surgical skin marker further comprising a handle, a marking agent, a marking tip extending forwardly of the handle and configured to apply a marking agent to the skin when contacted with the skin by a user, and a radiopaque pointer that is connected to the handle and proximally disposed relative to the marking tip;
placing the surgical skin marker in a position within a viewing field of an imaging screen of a radiographic imaging device, which position causes the radiopaque pointer to be proximal to the skin surface and aligned with and proximal to the marking tip; and
marking the skin as desired while viewing the skeletal elements to achieve a desired purpose.

21. The method of claim 20 wherein the desired purpose comprises marking the position of at least one bone, joint or skeletal anomaly appearing within the viewing field.

22. The method of claim 21 wherein the skeletal anomaly is selected from the group consisting of a fracture, dislocation or tumor.

23. The method of claim 20 wherein the desired purpose comprises identifying at least one surgical incision point or boundary on the skin surface.

24. The method of claim 20 wherein the desired purpose comprises identifying at least one skeletal element of interest while instructing students in a learning environment.

25. The method of claim 25 wherein the imaging screen is operatively connected to a radiographic imaging device selected from the group consisting of a radiographic imaging screen associated with a Zeiss Scan, an X-Ray machine, a C-Arm or a Mini C-Arm.
